(19) United States
(12) Patent Application Publication
(10) Pub. No.: US 2022/0254479 A1
(43) Pub. Date: Aug. 11, 2022

(54) SMART MEDICATION LABEL

(71) Applicant: HealthBeacon Limited, Dublin (IE)

(72) Inventors: James JOYCE, Needham, MA (US); Kieran DALY, Dublin (IE); Richard SHATTOCK, Dublin (IE)

(21) Appl. No.: 17/622,142

(22) PCT Filed: Jun. 26, 2019
(86) PCT No.: PCT/US2019/039280

§ 371 Date: Dec. 22, 2021

(51) Int. Cl. G16H 40/20; A61M 5/00; G16H 20/13

(52) U.S. Cl. CPC G16H 40/20; A61M 5/00; A61J 2205/10; A61J 2200/30; A61J 2205/60; G16H 20/13

(57) ABSTRACT

A system includes a medical item affixed with an electronically readable label, and a receptacle having a sensor configured to read the electronically readable label and communicate data pertaining to the electronically readable label with a server-based medical adherence application.

### BACKGROUND OF THE INVENTION

The present invention relates generally to medications, and more particularly to a smart medication label.

In general, self-injection devices or syringes allow patients to self-administer their medications outside the hospital, clinic or physician's office. These self-injection devices may be mechanical and include a pre-filled syringe which dispenses a medicine via needle after activation of a release button.

Many of these injectable devices are used for the management of chronic diseases often delivering expensive medicines. While methods to deliver drugs have improved, patient non-adherence to these medications is a growing concern for clinicians and Healthcare teams. Non-adherence to medications has adverse effects clinically and financially and impact already overburdened healthcare systems.

Patients prescribed these medications are typically provided a sharps container to dispose of used sharps conveniently and safely.

### BRIEF SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In general, in one aspect, the invention features a system including a medical item affixed with an electronically readable label, and a receptacle having a sensor configured to read the electronically readable label and communicate data pertaining to the electronically readable label with a server-based medical adherence application adapted to analyze, summarize, and profile the received data.

In another aspect, the invention features a method including providing a medical item tagged with an electronically readable label, providing a receptacle having a sensor configured to read the electronically readable label, reading the electronically readable label, and communicating data pertaining to the electronically readable label with a cloud-based medical adherence application.

Embodiments of the invention may have one or more of the following advantages.

The present invention can turn a passive syringe or auto-injector into a connected device through a connected sharps bin.

The present invention can provide a low cost connected medication system.

The present invention can provide an alternative to developing expensive connected auto-injectors or smart medication packaging

The present invention provides a connected hub, e.g., connected sharps bin, designed to work and communicate with the smart label on medical waste.

A smart label on medical waste generally requires lower energy requirements to connect a medication.

The present invention reduces the chances of connectivity issues.

The present invention can provide a process to aid in the training of patients who undergo injection training when they are first prescribed their medication.

The present invention can work in conjunction with electronic documents which provides data on a history of a particular batch of a drug.

The present invention can aid in drug brand authentication.

The present invention can aid in product quality assurance, e.g., medication shelf life.

The present invention can be used to train patients how to use their medication system, adhere the smart label (if required) and utilize their connected sharps bin to enhance their ability to self-administer their medication in a home setting and improve their medication self-management. Digital training modules can be incorporated to include a digital nurse or augmented reality nurse that can introduce the system in a series of training modules that includes reminders, notifications or interventions when appropriate or as set out in a nurse/patient digital visitation schedule. The digital nurse has the ability to update a patient when updates to the system happen (automatically), such as new functionality associated with the connected medication system.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

These and other features, aspects, and advantages of the present invention will become better understood with reference to the following description, appended claims, and accompanying drawings where:

FIG. 1 is a block diagram of an exemplary medical adherence device.

FIG. 2 is a block diagram of an exemplary syringe with an affixed electronically readable label.

### DETAILED DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

As described above, patients prescribed certain medications are typically provided a sharps container to dispose of used sharps conveniently and safely. In general, a sharps container is a hard plastic container that is used to safely dispose of hypodermic needles and other sharp medical instruments, such as IV catheters and disposable scalpels. Sharps containers may be single use or more typically, reusable. Needles are dropped into the sharps container through an opening in the top.

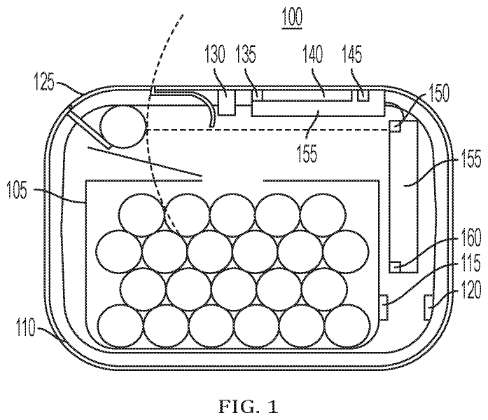

As shown in FIG. 1, in an embodiment, an exemplary medical adherence device 100 includes a sharps bin 105 positioned in an enclosure 110. Although this embodiment includes a separate sharps bin 105 and enclosure 110, other implementations can include some or all elements resident within a single unit.

The sharps bin 105 may include an identification device 115 such as a Bluetooth device, GPS device, near field communications card (NFC), bar code, QR code, RFID tag or other device. The sharps bin identification device 115 includes data specific to the sharps bin 105, such as an identification number or patient ID. In one embodiment, sharps bin identification device 115 is paired with an enclosure identification device 120 positioned in the enclosure 110. The enclosure identification device 120 can include, for example, a Bluetooth device, GPS device, NFC card reader, bar code reader, QR code reader, a RFID reader, and so forth.

In one embodiment, the sharps bin 105 is adapted to receive spent tagged medical syringes. In other embodiments, the sharps bin 105 is adapted to other types of tagged medical waste, such as spent tagged medicine bottles, tagged tubes, tagged containers, and so forth. More specifically, syringes and other types of medical waste include an affixed smart label that is referred herein as “smart label.” The smart label may include, for example, one or more of the following types of communication labels: radio/electronic inlays—single or dual frequency NFC/UHF, barcodes, QR codes, digital watermarks, RFID, and so forth. These labels can provide short and long range tracking. For example, short range (e.g., 1-4 cm) can be used to track consumer engagement, while long range (≥1 meter) can be used for prescription management/stock control.

Thus, the use of a electronically readable label (i.e., smart label) to tag the passive medication, syringe or injection pen enables tracking of the connected medication throughout an administration process and disposal through a localized proximity detection system.

The enclosure 110 includes at least a hinged door 125 controlled by a release latch 130. The enclosure 110 also can include one or more of a sensor 135, a display screen 140, such as a liquid crystal (LCD) display, a power indicator 145, such as a light emitting diode (LED) light, a camera 150, and one or more sim cards 155 for wireless communication (e.g., exchange of data) to one or more cloud-based medical adherence applications. The sensor 135 is configured to read a proximately located smart label. Thus, syringes and other types of medical waste that include a smart label are detected when being proximate to or passing by the sensor 135.

In some embodiments, the enclosure 110 includes a universal serial bus (USB) 160 connector.

The display screen 140 is adapted to program data and/or present data including, but not limited to, a status of the device 100 (e.g., full), a date, a position on a body of the patient where the patient should inject the medication, an adherence rating, and so forth.

In one embodiment, the release latch 130 opens the door 125 and activates the camera 150. In one embodiment, the door 125 includes a glass section that acts as a chamber for the syringe as an image is taken. In addition, the glass section may act as a safety mechanism so that a patient's hands are kept safe once a syringe is placed through the door 125. In still another embodiment, pressing on the door 125 activates the camera 150. The camera 150 remains activated until the door 125 closes, either manually or automatically. The camera 150 can include a sensor that stays active until a syringe passes through its field of view. Once the camera 150 detects a change in light representing the passage of the syringe through its field of view, or, in one specific implementation, the passing of the syringe through an infrared beam, an image is captured and stored, along with at least a time stamp. As soon as the image is captured, the camera 150 sends a signal to the door 125 to close. In addition, once the image is captured, information displayed on display device 140 is updated, reflecting real time patient habits. In one embodiment, the updated data includes an adherence score. The captured image and time stamp may be stored for further analysis and/or wirelessly transmitted by the one or more sim cards 155 to one or more cloud-based medical adherence applications.

The cloud-based medical adherence applications are adapted to analyze, summarize, and/or profile the received data for patient behaviors, possible medical interventions, and so forth. In addition, reports generated by the medical adherence applications may be used to support clinician and patient dialogue around adherence. In embodiments, the medical adherence applications present patient and clinician dashboards specifically targeted at a patient, a doctor and/or a pharmacist.

Elements of the medical adherence device 100 may be powered by an internal power supply located within the enclosure 110 or elsewhere. The internal power supply may be AC, DC or battery.

In summary, the medical adherence device 100 provides a system that communicates with a connected disposal bin, e.g., connected sharps bin. The connected disposal bin is provided to a patient as part of their medication prescription. This connected sharps container may be programmed with the patient's prescription schedule and notify the patient when to take their medication.

A smart label which communicates with the bin is applied to the patient's medication. For example, if the patient is on an injectable medication then their injectable device is labeled with the smart bin connector label which communicates with the smart sharps bin prior to and after administration and disposal. This enables the conversion of a non-connected medication to a connected medication via a connected disposal container for the purpose of assisting patients with management and verification of medications, prescription schedules and medication adherence habits, as well as the verification and subsequent management of waste disposal.

In addition, the system described above enables a unique pairing of a connected waste container with an injectable device or syringe.

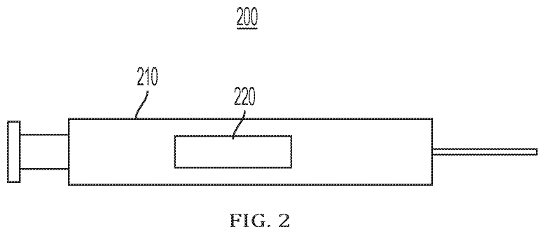

Tagging of medical waste involves affixing any type of electronically readable label on the medical waste. The electronically readable label may be, for example, a near field communications card (NFC), a bar code, a QR code, a RFID tag or other device. In FIG. 2, an exemplary syringe 200 includes a main body 210 and an electronically readable label 220 affixed to the main body 210 in such a manner the it can be detected by a sensor device in a sharps bin. More specifically, as described above, the sharps bin is adapted to read whichever electronically readable label 220 is affixed to the medical waste 200 as it passes into the sharps bin or other smart medical waste receptacle.

Thus, the present invention provides a complete medical adherence platform. More specifically, an injectable device or syringe is labeled with a smart label. The medication is administered by the patient and is brought into close proximity of the connected sharps container. The connected sharps container detects the smart labeled syringe or pen via short range communication technology. In two-step verification version, depositing of the pen or syringe into the sharps container also triggers a digital event recording the drop activity, which can also include a photographic image capture.

In the event that a drop event is expected based on the patient's injection or medication schedule but does not occur, this non-event can trigger a notification cascade and the notifications will be dependent on user interactions and the previous patient interaction and history of engaging with these connected tools.

The connected sharps bin also houses a disposable internal sharps container, into which drops the used sharps or medication packaging. The internal sharps container has a unique identifier (digital or analog) which is paired with the external smart sharps container. When this sharps container is full the internal sharps container can be swapped for a new empty bin. This event is registered and recorded and each internal bin is paired with the external bin and subsequently with any deposited medication waste.

In one embodiment, the smart tagged injectable device may house a mechanism for blocking or shielding the tag communication infrastructure. For example, the action of uncapping the injector pen before administration activates an internal communication signal which is then detected by the connected sharps container. Such a process enables all unshielded pens to be read by the connected sharps container and registered as used injectable devices following their unshrouding and disposal into the connected sharps container.

For inventory management within a patient's home setting, the tagged medication can be detected while in storage (ambient or cold storage) providing an indication of units available and can notify when a medication is running low. The disposal within the connected sharps container also allows a confirmation to manufacturers that a specific serial number has been disposed of and provides an electronic tracking mechanism for serialization requirements.

It would be appreciated by those skilled in the art that various changes and modifications can be made to the illustrated embodiments without departing from the spirit of the present invention. All such modifications and changes are intended to be within the scope of the present invention except as limited by the scope of the appended claims.

What is claimed:

1. A system comprising:
a medical item affixed with an electronically readable label; and
a receptacle having a sensor configured to read the electronically readable label and communicate data pertaining to the electronically readable label with a server-based medical adherence application adapted to analyze, summarize, and profile the received data.

2. The system of claim 1 wherein the medical item is a syringe or injectable device.

3. The system of claim 1 wherein the electronically readable label is Near Field Communication (NFC) card.

4. The system of claim 1 wherein the electronically readable label is a barcode.

5. The system of claim 1 wherein the electronically readable label is a QR code or AR code.

6. The system of claim 1 wherein the electronically readable label is a digital watermark.

7. The system of claim 1 wherein the electronically readable label is a RFID tag.

8. The system of claim 1 wherein the receptacle further comprises a camera configured to capture an image of the electronically readable label and communicate the captured image to the server-based medical adherence application.

9. The system of claim 1 wherein the receptacle further comprises a memory configured to receive patient medication information.

10. The system of claim 9 wherein the patient information includes dosage, date and time of recommended administration of the medical item.

11. The system of claim 10 wherein the server-based medical adherence application compares the patient information with the data pertaining to the electronically readable label.

12. The system of claim 11 wherein the comparison is communicated to a smartphone of a patient.

13. The system of claim 11 wherein the comparison is communicated to a healthcare provider.

14. A method comprising:
providing a medical item tagged with an electronically readable label;
providing a receptacle having a sensor configured to read the electronically readable label;
reading the electronically readable label; and
communicating data pertaining to the electronically readable label with a cloud-based medical adherence application.

15. The method of claim 4 wherein the cloud-based medical adherence application is configured to analyze, summarize, and profile the received data.

16. The method of claim 15 wherein the medical item is a syringe.

17. The method of claim 15 wherein the electronically readable label is selected from the group consisting of a NFC card, a barcode, a QR code, a digital watermark and a RFID tag.

18. The method of claim 17 further comprising comparing stored patient information with the data pertaining to the electronically readable label.

19. The method of claim 18 further comprising communicating the comparison to a smartphone of a patient.

20. The method of claim 18 further comprising communicating the comparison to a healthcare provider.

21. The method of claim 17 wherein the electronically readable label is shielded and detected by the receptacle having a sensor when uncoupled.

22. The method of claim 14 further comprising registering and reconciling the data with a patient's medical adherence application.

23. The method of claim 14 further comprising registering and reconciling the data with a patient's disposal adherence application.
